(19) United States
(12) Patent Application Publication
(10) Pub. No.: US 2026/0294971 A1
(43) Pub. Date: Oct. 1, 2026

(54) VIRAL VECTORS FOR CELL THERAPY

(71) Applicant: The Regents of the University of California, Oakland, CA (US)

(72) Inventors: Wendell A. LIM, San Francisco, CA (US); Ricardo BARBOSA DE ALMEIDA, San Francisco, CA (US); Milos SIMIC, San Francisco, CA (US); Gregory ALLEN, San Francisco, CA (US)

(21) Appl. No.: 19/480,739

(22) PCT Filed: May 2, 2024
(86) PCT No.: PCT/US2024/027562

§ 371 Date: Oct. 31, 2025

Related U.S. Application Data
(60) Provisional application No. 63/464,499, filed on May 5, 2023.

(51) Int. Cl. A61K 35/17; A61K 40/11; A61K 40/31; A61K 40/42; A61P 35/00; C07K 16/28; C12N 15/86

(52) U.S. Cl. CPC A61K 35/17; A61K 40/11; A61K 40/31; A61K 40/4204; A61P 35/00; C07K 16/28; C07K 16/2863; C12N 15/86; A61K 2239/13; A61K 2239/21; A61K 2239/22; A61K 2239/23; C12N 2740/15043; C12N 2840/203

(57) ABSTRACT

This disclosure provides, among other things, a recombinant nucleic acid comprising, in order from 5′ to 3′: (a) a first promoter, (b) a first coding sequence, wherein the first coding sequence encodes an therapeutic protein and is operably linked to the first promoter, (c) a second promoter; and (d) a second coding sequence, wherein the second coding sequence encodes a binding triggered transcriptional switch (BTTS) that is activated by binding to a cell surface antigen or protein in the extracellular matrix and is operably linked to the second promoter. Binding of the expressed BTTS to an antigen on the surface of a target cell or protein in the extracellular matrix activates expression of the therapeutic protein by the cytotoxic immune cell. Methods of treating various diseases, e.g., cancer, are also provided.

### CROSS-REFERENCING

This application claims the benefit of U.S. provisional application Ser. No. 63/464,499 filed on May 5, 2023, which application is incorporated by reference herein.

### GOVERNMENT RIGHTS

This invention was made with government support under U54 CA244438 awarded by the National Institutes of Health. The government has certain rights in the invention.

### REFERENCE TO AN ELECTRONIC SEQUENCE LISTING

The contents of the electronic sequence listing (UCSF-740WO_Seq_List.xml; Size: 11,350 bytes; and Date of Creation: May 2, 2024) is herein incorporated by reference in its entirety.

### INTRODUCTION

Despite enormous promise of CAR T cell therapy, some of the major challenges still facing this novel form of cancer therapy include accurately distinguishing cancer from healthy tissue and addressing the immunosuppressive tumor microenvironment (TME) to enhance T cell function. Next generation therapeutic cell designs, such as multi-antigen “prime and kill” therapeutic T cell circuits show great promise in tackling these challenges, but invariably require the ability to deliver multiple and larger transgenes to support engineering these novel therapeutic cells. A significant challenge is then to translate these more complex research grade multi gene designs and successfully adapt them into clinical grade vectors optimized for efficient large-scale manufacturing in the strict standards required for human use.

This disclosure is believed to address this problem.

### SUMMARY

This disclosure provides, among other things, a recombinant nucleic acid design that has been optimized for manufacturing of therapeutic T cells with multi-antigen “prime and kill” therapeutic circuits for treatment of various cancers, including solid tumors, and other diseases. This recombinant nucleic acid encodes two major components: (1) an inducible transgene encoding a therapeutic payload (such as an immune receptor or antibody, etc.) and (2) a constitutive transgene encoding a binding triggered transcriptional switch (BTTS) designed to sense a cell surface antigen or a protein in the extracellular matrix and upregulate expression of the inducible transgene when it is activated. The recombinant nucleic acid can be configured with both transgenes placed in tandem and in the sense orientation with a viral backbone, with the inducible transgene placed upstream of the constitutive transgene.

In some embodiments, the recombinant nucleic acid may comprise, in order from 5′ to 3′: (a) a first promoter, (b) a first coding sequence, wherein the first coding sequence encodes a therapeutic protein and is operably linked to the first promoter, (c) a second promoter; and (d) a second coding sequence, wherein the second coding sequence encodes a binding triggered transcriptional switch (BTTS) that is activated by binding to a cell surface antigen or a protein in the extracellular matrix and is operably linked to the second promoter. In these embodiments, transfection of the recombinant nucleic acid into a immune cell results in expression of the BTTS on the surface of the immune cell and binding of the expressed BTTS to an antigen on the surface of a target cell protein in the extracellular matrix activates expression of the therapeutic protein by the immune cell.

In some embodiments, a single vector encodes all components of the “prime and kill” BTTS multi-antigen therapeutic circuit. This design reduces both viral and cell manufacturing costs, improving viability for clinical application. In addition, the vector is compatible with current cGMP viral production methods, clinical manufacturing of therapeutic cells (e.g., T cells) and use in human patients. The design encodes protein binders optimized for biological activity and viral production, is devoid of potentially immunogenic protein tags, and is compatible with state-of-the-art 3^{rd} generation lentivirus system for improved efficacy and biosafety. Moreover, the configuration of the two transgenes enhances inducibility of the BTTS therapeutic circuit, while minimizing basal, ligand-independent activation of the circuit improving safety by minimizing on-target off-tumor potential for adverse effects. Finally, a single vector design requires fewer viral integration events when compared with multi-vector designs, improving patient safety by reducing the risk of insertional mutagenesis and genotoxicity.

Vectors and cells that contain the recombinant nucleic acid as well as methods of treating cancer using the cells are also provided.

Examples of such recombinant nucleic acids, cells and methods are described in further detail below.

### BRIEF DESCRIPTION OF THE DRAWINGS

FIGS. 1A-1C: Overview of E-SYNC therapeutic T cells and generation of p885 E-SYNC clinical single lentiviral transfer vector from the original two vector research-grade design. FIG. 1A: Diagram of “prime and kill” E-SYNC T cell circuit. FIGS. 1B, 1C: Illustrations of constructs used.

FIGS. 2A-2C: Examples of single lentiviral vector configurations for BTTS driven CAR circuits. Several different lentiviral vector configurations are shown, where transgenes are either placed in tandem (FIGS. 2A and 1B) or in divergent orientation (FIG. 2C).

FIGS. 3A-3B: E-SYNC T cells prepared with p885 single vector show EGFRvIII-driven CAR expression and kill GBM tumor cells in vitro similarly to reference dual vector cells. FIG. 3A. P885 T cells can activate CAR expression in response to EGFRvIII exposure to similar levels as dual vector reference cells while maintaining low basal CAR expression in the absence of stimulation. FIG. 3B. Killing assay measuring the capability of CD8+ T cells transduced with P168/322, P830 or P885 of clearing GBM cells shows that single vector designs can match dual vector reference.

FIG. 4: E-SYNC T cells prepared with p885 clinical vector are effective in clearing EGFRvIII+ GBM patient derived xenograft brain tumors in mice.

FIGS. 5A and 5B: E-SYNC T cells transduced with p885 single vector can discriminate between brain vs. flank tumors in vivo based on tumor EGFRvIII expression. Model is shown in FIG. 5A. Results are shown in FIG. 5B.

FIGS. 6A-6C: Overview of BCAN-primed SynNotch-CAR circuit (B-SYNC), composition of pB-SYNC1 single lentiviral transfer vector and tumor cell killing performance of B-SYNC T cells. FIG. 6A. Overview of pB-SYNC1 vector design to produce BCAN-primed SynNotch-CAR (B-SYNC) T cells. FIG. 6B. Diagram of B-SYNC therapeutic T cell. FIG. 6C. T cells transduced with pB-SYNC1 single vector outperform dual vector reference cells in this in vitro co-culture killing assay of GBM6 PDX.

FIG. 7: B-SYNC T cells prepared with pB-SYNC1 clinical vector are effective in clearing GBM patient derived xenograft tumors in mice and improving survival.

FIGS. 8A and 8B: Plasmid maps for p885 (FIG. 8A) and pB-SYNC1 (FIG. 8B) vectors.

FIGS. 9A-9E: Single vector design variants with BTTS controlled expression of multiple protein payloads. FIG. 9A. The tandem design for a single CAR payload. FIG. 9B. A tandem vector design. FIG. 9C. Another alternative design having a bi-directional promoter. FIGS. 9D and 9E show data obtained from the constructs.

FIGS. 10A and 10B: Modifying 5′ LTR to use CMV promoter enhances titer of large vector designs using state of the art 3rd generation lentiviral system for clinical use. FIG. 10A show replacing the promoter in the 5′ LTR with a CMV promoter. FIG. 10B: shows that the CMV-5′ LTR outperforms the RSV-5′ LTR in functional titer and T cell transduction.

### DEFINITIONS

As used herein, the terms “treatment,” “treating,” “treat” and the like, refer to obtaining a desired pharmacologic and/or physiologic effect and/or a response related to the treatment. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” refers to the amount of an agent (including biologic agents, such as cells), or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “individual,” “subject,” “host,” and “patient,” used interchangeably herein, refer to a mammal, including, but not limited to, murines (e.g., rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

As used herein, the term “binding-triggered transcriptional switch” or “BTTS” refers to any polypeptide or complex of the same that is capably of transducing a specific binding event on the outside of the cell (e.g., binding of an extracellular domain of the BTTS) to activation of a recombinant promoter within the nucleus of the cell. Many BTTSs work by releasing a transcription factor that activates the promoter. In these embodiments, the BTTS is made up of one or more polypeptides that undergo proteolytic cleavage upon binding to the antigen to release a gene expression regulator that activates the recombinant promoter. For example, a BTTS may comprise (i) an extracellular domain comprising the antigen binding region of an antigen-specific antibody; (ii) a proteolytically cleavable sequence comprising one or more proteolytic cleavage sites; and (iii) an intracellular domain, wherein binding of the antigen binding region to the antigen induces cleavage of the sequence at the one or more proteolytic cleavage sites, thereby releasing the intracellular domain and wherein the intracellular domain activates transcription of an expression cassette. A BTTS can be based on Notch, A2, MESA, or a force receptor, for example, although others are known (e.g., see Zhu et al 2022 Cell. 185: 1431-1443, US20200331985 and WO2021061856).

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional heterodimerization of the two portions of the split CAR is pharmacologically controlled (e.g., as described in PCT publication no. WO 2014/127261 A1 and US Patent Application No. 2015/0368342 A1, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

The terms “T cell receptor” and “TCR” are used interchangeably and will generally refer to a molecule found on the surface of T cells, or T lymphocytes, that is responsible for recognizing fragments of antigen as peptides bound to major histocompatibility complex (MHC) molecules. The TCR complex is a disulfide-linked membrane-anchored heterodimeric protein normally consisting of the highly variable alpha (α) and beta (β) chains expressed as part of a complex with CD3 chain molecules. Many native TCRs exist in heterodimeric αβ or γδ forms. The complete endogenous TCR complex in heterodimeric ac form includes eight chains, namely an alpha chain (referred to herein as TCRα or TCR alpha), beta chain (referred to herein as TCRβ or TCR beta), delta chain, gamma chain, two epsilon chains and two zeta chains. In some instance, a TCR is generally referred to by reference to only the TCRα and TCRβ chains, however, as the assembled TCR complex may associate with endogenous delta, gamma, epsilon and/or zeta chains an ordinary skilled artisan will readily understand that reference to a TCR as present in a cell membrane may include reference to the fully or partially assembled TCR complex as appropriate. Recombinant or engineered individual TCR chains and TCR complexes have been developed. References to the use of a TCR in a therapeutic context may refer to individual recombinant TCR chains. As such, engineered TCRs may include individual modified TCRα or modified TCRβ chains as well as single chain TCRs that include modified and/or unmodified TCRα and TCRβ chains that are joined into a single polypeptide by way of a linking polypeptide.

The terms “synthetic”, “chimeric” and “engineered” as used herein generally refer to artificially derived polypeptides or polypeptide encoding nucleic acids that are not naturally occurring. Synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods. Chimeric and engineered polypeptides or polypeptide encoding nucleic acids will generally be constructed by the combination, joining or fusing of two or more different polypeptides or polypeptide encoding nucleic acids or polypeptide domains or polypeptide domain encoding nucleic acids. Chimeric and engineered polypeptides or polypeptide encoding nucleic acids include where two or more polypeptide or nucleic acid “parts” that are joined are derived from different proteins (or nucleic acids that encode different proteins) as well as where the joined parts include different regions of the same protein (or nucleic acid encoding a protein) but the parts are joined in a way that does not occur naturally.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

A “vector” or “expression vector” is a replicon, such as plasmid, phage, virus, or cosmid, to which another DNA segment, i.e. an “insert”, may be attached so as to bring about the replication of the attached segment in a cell.

The term “heterologous”, as used herein, means a nucleotide or polypeptide sequence that is not found in the native (e.g., naturally-occurring) nucleic acid or protein, respectively. Heterologous nucleic acids or polypeptide may be derived from a different species as the organism or cell within which the nucleic acid or polypeptide is present or is expressed. Accordingly, a heterologous nucleic acids or polypeptide is generally of unlike evolutionary origin as compared to the cell or organism in which it resides.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the cell” includes reference to one or more cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As noted above, this disclosure provides a recombinant nucleic acid comprising, in order from 5′ to 3′: (a) a first promoter, (b) a first coding sequence, wherein the first coding sequence encodes a therapeutic protein and is operably linked to the first promoter, (c) a second promoter; and (d) a second coding sequence, wherein the second coding sequence encodes a binding triggered transcriptional switch (BTTS) that is activated by binding to a cell surface antigen or a protein in the extracellular matrix and is operably linked to the second promoter. In these embodiments, transfection of the recombinant nucleic acid into a immune cell results in expression of the BTTS on the surface of the immune cell and binding of the expressed BTTS to an antigen on the surface of a target cell or protein in the extracellular matrix activates expression of the therapeutic protein by the immune cell. Transfection of the recombinant nucleic acid (e.g., via a viral vector) into a immune cell results in expression of the BTTS on the surface of the immune cell and binding of the expressed BTTS to an antigen on the surface of a target cell or protein in the extracellular matrix activates expression of the therapeutic protein by the immune cell.

In any embodiment, the immune cell may be a cytotoxic immune cell, e.g., a cytotoxic T cell.

The therapeutic protein may be an immune receptor such as a CAR or TCR, an antibody, a cytokine, a cytokine receptor or an enzyme, for example. In some embodiments, the therapeutic protein, when expressed, may be secreted by the cell or may be on the surface of the cell. In embodiments in which the therapeutic protein is secreted, the therapeutic protein may be, for example, an antibody (e.g., an antibody that binds to PD1, PD-L1, PD-L2, CTLA4, TIM3 or LAG3 or another immune checkpoint, for example), an enzyme (e.g., a superoxide dismutase for removing reactive oxygen species or a protease that can unmask a probody) or a bioactive peptide such as a cytokine (e.g., Il-1ra, IL-4, IL-6, IL-10, IL-11, IL-13, or TGF-β, among many others).

The disclosures of WO2019195586, US20200331985 and Choe et al (Sci. Transl. Med. 2021 13: eabe7378) are expressly incorporated by reference herein for all purposes.

In any embodiment, the cytotoxic immune cell does not comprise an anti-EphA2/IL13Rα2 tandem CAR and a BTTS that is activated by binding to EGFRvIII or a brain-specific marker such as BCAN.

### Constructs for Expressing Multiple Payloads

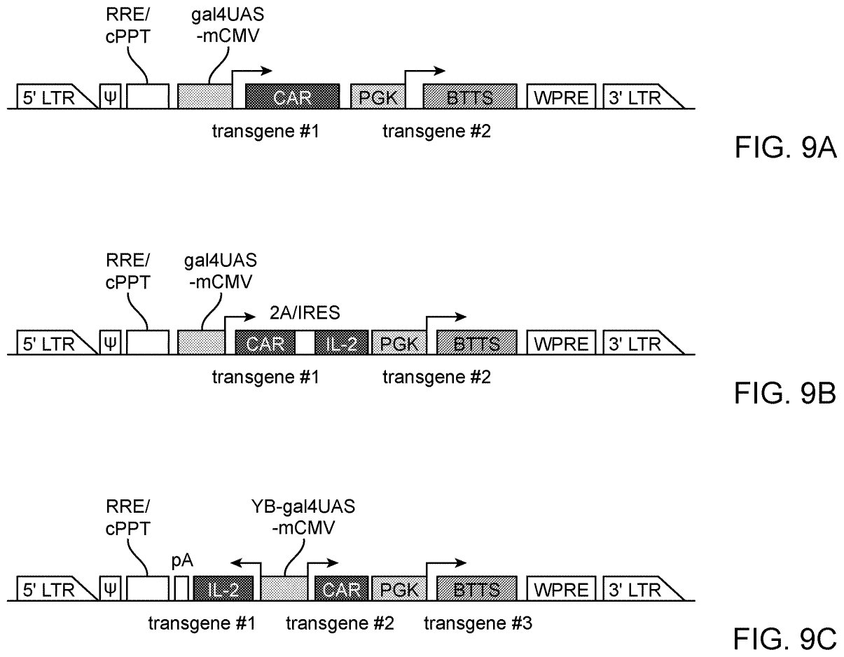

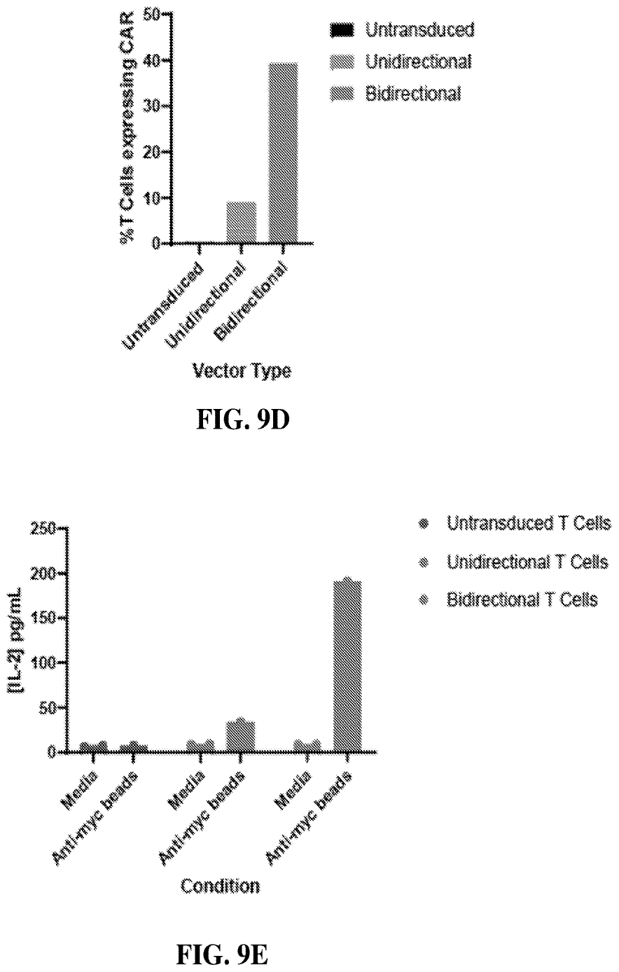

Additional embodiments are shown in FIG. 9A-C. In these embodiments, a third protein can be encoded by the construct using an IRES, a protease cleavage site or a bidirectional promoter. In these embodiments, the expression of the proteins encoded by the first and third coding sequences may be activated by the BTTS.

In these embodiments, the construct may encode a BTTS, an immune receptor such as a CAR and a cytokine that will be secreted from the cell. In these embodiments, the circuit may comprise a nucleic acid containing a promoter that is activated by the released transcriptional activator, and coding sequence encoding the immune receptor and a pro-inflammatory protein, where expression the immune receptor and pro-inflammatory cytokine are activated by the BTTS binding to its target. In this disclosure, the term “pro-inflammatory protein” is intended to encompass any cytokine that have a pro-inflammatory activity (e.g., IL-2, CCL-21, IL-12, IL-7, IL-15 and IL-21, etc.), as well as non-natural or “engineered” cytokines that have pro-inflammatory activity such as super IL-2 (see, e.g., Levin et al Nature 2012 484: 529-533, which has the following amino acid substitutions L80F, R8ID, L85V, I86V, and I92F relative to wild type), mini-TGF-Beta (which blocks TGF-Beta signaling) and DR-18 (an IL-18 variant), etc.). Engineered cytokines include superkines, which often have up to 10 amino acid substitutes relative to a natural cytokine, as well as natural cytokines that have been truncated, and dominant variants. Cytokines of interest include selected from IL-2, IL-12, IL-15, IL-7, CD40L, or a non-natural variant of IL-2, IL-12, IL-15, IL-7, CD40L that has pro-inflammatory activity. Cytokines include “ortho” cytokines that can be paired with a receptor in the immune cell (see, e.g., Sockolosky et al. 2018). Other pro-inflammatory proteins include immune checkpoint inhibitors, including molecules that block interactions with PD1, CTLA4, BTLA, CD160, KRLG-1, 2B34, Lag-3, Tim-3 and other immune checkpoints. See, e.g., Odorizzi and Wherry (2012) J. Immunol. 188:2957; and Baitsch et al. (2012) PLoSOne 7: e30852. For example, an anti-PD1 antibody, an anti-PDL1 antibody a decoy resistant IL-18 and a dominant negative TGF-4, a dominant negative TGFβ receptor, or a TGF inhibitor/agonist could be used.

Sources for exemplary pro-inflammatory proteins are listed below.

| Payload | Exemplary sources of payload sequences |
|---|---|
| IL2 | NCBI Gene ID: 3558 |
| super-2 | Levin et al. Nature. 2012 Apr. 26; 484(7395): |
| | 529-533. |
| ortho2 | Sockolosky et al. Science. 2018 Mar. 2; 359(6379): |
| | 1037-1042. |
| IL15 | NCBI Gene ID: 3600 |
| Anti-PD1 | Pembrolizumab, Nivolumab, Cemiplimab, |
| | Dostarlimab, JTX-4014, Spartalizumab, |
| | Camrelizumab, Sintilimab, Tislelizumab, Toripalimab, |
| | INCMGA00012, AMP-224, AMP-514 |
| Anti-PDL1 | Atezolizumab, Avelumab, Durvalumab, KN035, |
| | CK-301, AUNP12, CA-170, BMS-986189 |
| IL7 | NCBI Gene ID: 3574 |
| CD40L | NCBI Gene ID: 959 |
| Decoy Resistant | Zhou et al. Nature. 2020 July; 583(7817): 609-614. |
| IL-18 (DRIL-18) | |
| Dominant | Kloss et al. Mol Ther. 2018 Jul. 5; 26(7): 1855-1866., |
| negative TGFb | Wieser et al. Mol Cell Biol. 1993 Dec; 13(12): |
| | 7239-47. |

In some embodiments, the cytokine could be an anti-inflammatory cytokine.

In some embodiments and as illustrated in FIG. 9C, expression of two proteins may be coordinated by a bidirectional promoter containing, e.g., one or more binding sites for the transcriptional activator that is cleaved from the BTTS (e.g., Gal4, TetO, LexA, or HNF1a sequences, among others), flanked by two minimal promoters (which may be selected from the minCMV, YB_TATA, T11, minIL2, minTK promoters, among others) in divergent orientation. In one example, the bidirectional promoter may contain a gal4 DNA binding site array (GAL4 UAS) flanked by two minimal promoters (e.g., the minimal CMV promoter (see, e.g., Heang et al, Mol Cell Bio. 1984 4: 2214-23) and the YB_TATA synthetic minimal promoter (see, e.g., Hansen et al, Proc. Natl. Acad. Sci. 2014 111: 15705-10) in divergent orientation.

The sequence of an example of a bidirectional promoter containing a gal4 DNA binding site array (GAL4 UAS) flanked by the minimal CMV promoter the YB_TATA synthetic minimal promoter in divergent orientation is set forth below:

(SEQ ID NO: 9)
GATGAGCTCTGGTAGTAGACAAGTGGCCCCCATTATATACCCTCTAGAA
CTAGTAATTCGGAGCACTGTCCTCCGAACGTCGGAGCACTGTCCTCCGA
ACGTCGGAGCACTGTCCTCCGAACGTCGGAGCACTGTCCTCCGAACGGA
GCATGTCCTCCGAACGTCGGAGCACTGTCCTCCGAACGACTAGTTAGGC
GTGTACGGTGGGAGGCCTATATAAGCAGAGCTCGTTTAGTGAACCGTCA
GATCGCCTGGAGACGCCATCCACGCTGTTTTGACCTCCATAGAAGACAC
CGGGACCGATCCAGC

In this sequence, the YB-TATA promoter is defined by the complement of positions 1-48 (which is composed of four segments: 1-25 (downstream spacer), 26-32 (flanker), 33-39 (TATA box) and 40-48 (flanker)), the Gal4 array is defined by positions 60-185, and the minimal CMV (mCMV) promoter is defined by positions 192-309. As such, in any embodiment the recombinant nucleic acid may comprise a bidirectional promoter that has at least 90%, at least 95% or 100% sequence identity to SEQ ID NO: 9.

In these embodiments, the first promoter may be bidirectional, the recombinant nucleic acid further may comprise a third coding sequence that is 5′ of the bi-directional promoter (i.e., a complement of a coding sequence or, alternatively, a coding sequence that is on the opposite strand), and binding of the BTTS to an antigen on the surface of a target cell or protein in the extracellular matrix activates expression of the proteins encoded by first and third coding sequences by the immune cell.

In some embodiments, the recombinant nucleic acid may further comprises a third coding sequence that is 3′ of the first coding sequence, before the second promoter, wherein the first and third coding sequences may separated by an IRES or protease cleavage site and binding of the BTTS to an antigen on the surface of a target cell or protein in the extracellular matrix activates expression of the proteins encoded by first and third coding sequences by the immune cell.

In either of these embodiments, the first coding sequence and the third coding sequence may encode a CAR and a pro-inflammatory cytokine, e.g., IL-2 (either respectively or not). In these embodiments the BTTS may in some cases recognize a tissue-specific marker (which may be a cell surface marker or a protein in the extracellular matrix), and the CAR may be activated by a marker that is expressed on cancer cells.

In embodiments, that contain a bidirectional promoter and a third coding sequence, the nucleic acid may also contain a transcriptional terminator for the third coding sequence, which should be 5′ of the complement of the third coding sequence. This element is believed to antisense gene expression. However, the selection of polyA (pA) site may impact lentiviral titers. In many cases, it may be important to select a unidirectional terminator that stops transcription in the antisense direction and enhances gene expression, without affecting proviral transcription in the sense direction. hGHpA was identified as being effective for both antisense gene expression and minimal impact on viral titer. However, other terminators may be used.

The complement of the hGHpA terminator (i.e., the bottom strand of the construct) may have the following sequence:

(SEQ ID NO: 10)
AGGACAGGGAAGGGAGCAGTGGTTCACGCCTGTAATCCCAGCAATTTGG
GAGGCCAAGGTGGGTAGATCACCTGAGATTAGGAGTTGGAGACCAGCCT
GGCCAATATGGTGAAACCCCGTCTCTACCAAAAAAACAAAAATTAGCTG
AGCCTGGTCATGCATGCCTGGAATCCCAACAACTCGGGAGGCTGAGGCA
GGAGAATCGCTTGAACCCAGGAGGCGGAGATTGCAGTGAGCCAAGATTG
TGCCACTGCACTCCAGCTTGGTTCCCAATAGACCCCGCAGGCCCTACAG
GTTGTCTTCCCAACTTGCCCCTTGCTCCATACCACCCCCCTCCACCCCA
TAATATTATAGAAGGACACCTAGTCAGACAAAATGATGCAACTTAATTT
TATTAGGACAAGGCTGGTGGGCACTGGAGTGGCAACTTCCAGGGCCAGG
AGAGGCACTGGGGAGGGGTCACAGGGATGCCACCC.

As such, in any embodiment the recombinant nucleic acid may comprise a terminator that has at least 90%, at least 95% or 100% sequence identity to the complement of SEQ ID NO: 10.

### Chimeric LTR/CMV Promoter Fusions

Also provided is a chimeric LTR/CMV promoter fusion which may be included in the present construct or other constructs. This sequence enhances viral titer and packaging efficiency and has the sequence of SEQ ID NO: 8.

In this sequence, nucleotides 25-404 and 405-607 are the CMV enhancer and promoter respectively, and nucleotides 622-802 are the truncated 5′ long terminal repeat (LTR) from HIV-1. As such, this disclosure provides a new LTR/CMV promoter fusion that has at least 90%, at least 95% or 100% sequence identity to SEQ ID NO: 8.

### Chimeric Antigen Receptors (CARs)

As noted above, the first coding sequence may encode a CAR. Binding of the CAR to its cognate antigen activates the immune cell. CARs can be designed in several ways (see, generally, e.g., Guedan et al, Methods and Clinical Development 2019 12: 145-156) and may include an extracellular domain that contains an antigen binding domain such as a scFv, nanobody or ligand, a hinge, a transmembrane region (which may be derived from CD4, CD8a, or CD28), a costimulatory signaling domains (which may be derived from the intracellular domains of the CD28 family (e.g., CD28 and ICOS) or the tumor necrosis factor receptor (TNFR) family of genes (e.g., 4-1BB, OX40, or CD27), and an ITAM domain, e.g., the signaling domain from the zeta chain of the human CD3 complex (CD3zeta). In practice, any of these domains may be a variation of a wild type sequence. In practice, any of these sequences may be a variant of a wild type sequence, e.g., a sequence that is at least 90%, 95, or 98% identical a sequence described in WO2014127261, for example.

In any embodiment, CAR may contain at least one immunoreceptor tyrosine-based activation motif (ITAM). In these embodiments, the ITAM (immunoreceptor tyrosine-based activation motif) may be described by the formula YX₁X₂L/I, where X₁ and X₂ are independently any amino acid. In some cases, the intracellular signaling domain of a subject engineered immune receptor comprises 1, 2, 3, 4, or 5 ITAM motifs. In some cases, an ITAM motif is repeated twice in an intracellular signaling domain, where the first and second instances of the ITAM motif are separated from one another by 6 to 8 amino acids, e.g., (YX₁X₂L/I)(X₃)ₙ(YX₁X₂L/I), where n is an integer from 6 to 8, and each of the 6-8 X₃ can be any amino acid. In some cases, the intracellular signaling domain of a subject engineered immune receptor comprises 3 ITAM motifs. Suitable ITAMs can be derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12; FCER1G (Fc epsilon receptor I gamma chain); CD3D (CD3 delta); CD3E (CD3 epsilon); CD3G (CD3 gamma); CD3Z (CD3 zeta); and CD79A (antigen receptor complex-associated protein alpha chain), although in practice functional variants of these domains (e.g., domains that have at least 90% or 95% sequence identify to a wild type domain) can be used in many cases.

### T Cell Receptors

Useful TCRs include essentially any TCR useful in the treatment of cancer, including single-chain and multi-chain TCRs, directed to a targeting antigen. A TCR used in the instant methods will generally include, at a minimum, an antigen binding domain and a modified or unmodified TCR chain, or portion thereof, including but not limited to e.g., a modified or unmodified α-chain, a modified or unmodified β-chain, etc. An employed TCR may further include one or more costimulatory domains. In some instances, a TCR employed herein will include an alpha chain and a beta chain and recognize antigen when presented by a major histocompatibility complex.

Essentially any TCR can be induced by a BTTS using a method of the present disclosure including e.g., TCRs that are specific for any of a variety of epitopes, including, e.g., an epitope expressed on the surface of a cancer cell, a peptide-MHC complex on the surface of cancer cell, and the like. In some cases, the TCR is an engineered TCR.

Non-limiting examples of engineered TCRs, including those having immune cell activation function and that may be modified to include an antigen-binding domain specific for a suitable targeting antigen, useful in the methods described herein include, e.g., antigen-specific TCRs, Monoclonal TCRs (MTCRs), Single chain MTCRs, High Affinity CDR2 Mutant TCRs, CD1-binding MTCRs, High Affinity NY-ESO TCRs, VYG HLA-A24 Telomerase TCRs, including e.g., those described in PCT Pub Nos. WO 2003/020763, WO 2004/033685, WO 2004/044004, WO 2005/114215, WO 2006/000830, WO 2008/038002, WO 2008/039818, WO 2004/074322, WO 2005/113595, WO 2006/125962; Strommes et al. Immunol Rev. 2014; 257(1):145-64; Schmitt et al. Blood. 2013; 122(3):348-56; Chapuls et al. Sci Transl Med. 2013; 5(174):174ra27; Thaxton et al. Hum Vaccin Immunother. 2014; 10(11):3313-21 (PMID:25483644); Gschweng et al. Immunol Rev. 2014; 257(1):237-49 (PMID:24329801); Hinrichs et al. Immunol Rev. 2014; 257(1):56-71 (PMID:24329789); Zoete et al. Front Immunol. 2013; 4:268 (PMID:24062738); Marr et al. Clin Exp Immunol. 2012; 167(2):216-25 (PMID:22235997); Zhang et al. Adv Drug Deliv Rev. 2012; 64(8):756-62 (PMID:22178904); Chhabra et al. Scientific World Journal. 2011; 11:121-9 (PMID:21218269); Boulter et al. Clin Exp Immunol. 2005; 142(3):454-60 (PMID:16297157); Sami et al. Protein Eng Des Sel. 2007; 20(8):397-403; Boulter et al. Protein Eng. 2003; 16(9):707-11; Ashfield et al. IDrugs. 2006; 9(8):554-9; Li et al. Nat Biotechnol. 2005; 23(3):349-54; Dunn et al. Protein Sci. 2006; 15(4):710-21; Liddy et al. Mol Biotechnol. 2010; 45(2); Liddy et al. Nat Med. 2012; 18(6):980-7; Oates, et al. Oncoimmunology. 2013; 2(2):e22891; McCormack, et al. Cancer Immunol Immunother. 2013 April; 62(4):773-85; Bossi et al. Cancer Immunol Immunother. 2014; 63(5):437-48 and Oates, et al. Mol Immunol. 2015 October; 67(2 Pt A):67-74; the disclosures of which are incorporated herein by reference in their entirety.

Useful TCRs include those having wild-type affinity for their respective antigen as well as those having enhanced affinity for their respective antigen. TCRs having enhanced affinity for their respective antigen may be referred to as “affinity enhanced” or “enhanced affinity” TCRs. The affinity of a TCR may be enhanced by any convenient means, including but not limited to binding-site engineering (i.e., rational design), screening (e.g., TCR display), or the like. Non-limiting examples of affinity enhanced TCRs and methods of generating enhanced affinity TCRs include but are not limited to e.g., those described in PCT Pub. Nos. 20150118208, 2013256159, 20160083449; 20140349855, 20100113300, 20140371085, 20060127377, 20080292549, 20160280756, 20140065111, 20130058908, 20110038842, 20110014169, 2003276403 and the like; the disclosures of which are incorporated herein by reference in their entirety. Further engineered TCRs, modified to be directed to an appropriate targeting antigen, that may be expressed in response to release of an intracellular domain of a BTTS of the present disclosure include e.g., those described in PCT Application No. US2017/048040; the disclosure of which is incorporated herein by reference in its entirety.

Useful TCRs, which may be modified to be directed to an appropriate targeting antigen, may, in some instances, also include those described in U.S. Pat. Nos. 9,889,161; 9,889,160; 9,868,765; 9,862,755; 9,717,758; 9,676,867; 9,409,969; 9,115,372; 8,951,510; 8,906,383; 8,889,141; 8,722,048; 8,697,854; 8,603,810; 8,383,401; 8,361,794; 8,283,446; 8,143,376; 8,003,770; 7,998,926; 7,666,604; 7,456,263; 7,446,191; 7,446,179; 7,329,731; 7,265,209; and 6,770,749; the disclosures of which are incorporated herein by reference in their entirety.

As described above, in some instances, the antigen binding domain of a TCR, such as but not limited to e.g., those described or referenced above, may be substituted or amended with an alternative or additional antigen binding domain directed to a different antigen, such as but not limited to one or more of the antigens described herein, for use in the herein described methods. In such instances, the other portions (i.e., the transmembrane domain, any intracellular signaling domains, etc.) of the antigen-domain-substituted TCR may or may not be modified.

### Binding-Triggered Transcriptional Switches (BTTSs)

The BTTS is a cleavable fusion protein contains: (a) an extracellular binding domain comprising a protein binding domain (e.g., scFv or nanobody) that binds to a cell surface antigen or a protein in the extracellular matrix, (b) a transmembrane domain, and (c) an intracellular domain comprising a transcriptional activator, where binding of the binding domain to the marker on the surface of another cell or a protein in the extracellular matrix induces proteolytic cleavage of the BTTS to release the transcriptional activator. In some embodiments, the BTTs may additional contain an extracellular force sensing region between regions (a) and (b) and (d) one or more force-dependent cleavage sites in (c) that are cleaved when the force sensing region is activated.

In this switch, the fusion protein is cleaved to release the intracellular domain when the extracellular domain of the fusion protein engages with a marker on another cell. As such, in many cases, the fusion protein will contain a sensing region (which is typically in the extracellular domain) and one or more cleavage sites that are cleaved when the sensing region is activated. The position of the cleavage sites may vary and, in some embodiments the fusion protein may contain at least two cleavage sites. In some cases, one of the cleavage sites may be extracellular and the other may be in the transmembrane domain or within 10 amino acids of the transmembrane domain in the intracellular domain. In any embodiment, the sensing region and/or the one or more cleavage sites may be from a Delta/Serrate/Lag2 (DSL) superfamily protein, as reviewed by Pintar et al (Biology Direct 2007 2: 1-13). For example, the sensing region and/or the one or more cleavage sites may be from Notch (see Morsut Cell. 2016 164: 780-91), von Willebrand Factor (vWF), amyloid-beta, CD16, CD44, Delta, a cadherin, an ephrin-type receptor or ephrin ligand, a protocadherin, a filamin, a synthetic E cadherin, interleukin-1 receptor type 2 (IL1R2), major prion protein (PrP), a neuregulin or an adhesion-GPCR. Several other examples of this type of protein are known and listed in Pintar, supra. Many members of this family appear to share a similar architecture a region that unfolds and opens up a protease cleavage site (e.g., EGF-like repeats; see Cordle et al Nat. Struct. Mol. Biol. 2008 15: 849-857), a trans-membrane segment, and a relatively short (~100-150 amino acids) intracellular domain. These sequences permit the binding-triggered release of a transcriptional activator from the membrane in their natural environment and can be readily adapted herein.

In some cases, the one or more ligand-inducible proteolytic cleavage sites are selected from S1, S2, and S3 proteolytic cleavage sites. In some cases, the S1 proteolytic cleavage site is a furin-like protease cleavage site comprising the amino acid sequence Arg-X-(Arg/Lys)-Arg, where X is any amino acid. In some cases, the S2 proteolytic cleavage site ADAM-17-type protease cleavage site comprising an Ala-Val dipeptide sequence. In some cases, the S3 proteolytic cleavage site is a γ-secretase cleavage site comprising a Gly-Val dipeptide sequence. The S3 proteolytic cleavage site is in the transmembrane domain. In some cases, the shear force generated by binding of the extracellular domain of this fusion protein to another cell or the extracellular matrix unfolds the force sensing region (which, in the case of Notch contains EGF-like repeats whereas in other protein is made up of other sequences such as the A2 domain in vWF (see, e.g., J Thromb Haemost. 2009 7:2096-105, Lippok Biophys J. 2016 110: 545-54, Lynch Blood. 2014 123: 2585-92, Crawley, Blood. 2011 118:3212-21 and Xy J Biol Chem. 2013 288:6317-24) or modified A2 domain that has, e.g., the R1597W, E1638K and I1628T substitutions. The architecture of such proteins is described in, e.g., Morsut et al, Cell. 2016 164: 780-91, WO2016138034 and WO2019099689, among other places).

In some cases, the fusion protein includes an S1 ligand-inducible proteolytic cleavage site. An S1 ligand-inducible proteolytic cleavage site can be located between the HD-N segment and the HD-C segment. In some cases, the S1 ligand-inducible proteolytic cleavage site is a furin-like protease cleavage site. A furin-like protease cleavage site can have the canonical sequence Arg-X-(Arg/Lys)-Arg, where X is any amino acid; the protease cleaves immediately C-terminal to the canonical sequence. For example, in some cases, an amino acid sequence comprising an S1 ligand-inducible proteolytic cleavage site can have the amino acid sequence GRRRRELDPM (SEQ ID NO: 1), where cleavage occurs between the “RE” sequence. As another example, an amino acid sequence comprising an S1 ligand-inducible proteolytic cleavage site can have the amino acid sequence RQRRELDPM (SEQ ID NO: 2), where cleavage occurs between the “RE” sequence.

In some cases, the fusion protein polypeptide includes an S2 ligand-inducible proteolytic cleavage site. An S2 ligand-inducible proteolytic cleavage site can be located within the HD-C segment. In some cases, the S2 ligand-inducible proteolytic cleavage site is an ADAM-17-type protease cleavage site. An ADAM-17-type protease cleavage site can comprise an Ala-Val dipeptide sequence, where the enzyme cleaves between the Ala and the Val. For example, in some cases, amino acid sequence comprising an S2 ligand-inducible proteolytic cleavage site can have the amino acid sequence KIEAVKSE (SEQ ID NO: 3), where cleavage occurs between the “AV” sequence. As another example, an amino acid sequence comprising an S2 ligand-inducible proteolytic cleavage site can have the amino acid sequence KIEAVQSE (SEQ ID NO: 4), where cleavage occurs between the “AV” sequence.

In some cases, the fusion protein includes an S3 ligand-inducible proteolytic cleavage site. An S3 ligand-inducible proteolytic cleavage site can be located within the TM domain. In some cases, the S3 ligand-inducible proteolytic cleavage site is a gamma-secretase (γ-secretase) cleavage site. A γ-secretase cleavage site can comprise a Gly-Val dipeptide sequence, where the enzyme cleaves between the Gly and the Val. For example, in some cases, an S3 ligand-inducible proteolytic cleavage site has the amino acid sequence VGCGVLLS (SEQ ID NO: 5), where cleavage occurs between the “GV” sequence. In some cases, an S3 ligand-inducible proteolytic cleavage site comprises the amino acid sequence GCGVLLS (SEQ ID NO: 6).

In some cases, the fusion protein polypeptide lacks an S1 ligand-inducible proteolytic cleavage site. In some cases, the BTTS lacks an S2 ligand-inducible proteolytic cleavage site. In some cases, the BTTS lacks an S3 ligand-inducible proteolytic cleavage site. In some cases, the BTTS lacks both an S1 ligand-inducible proteolytic cleavage site and an S2 ligand-inducible proteolytic cleavage site. In some cases, the BTTS includes an S3 ligand-inducible proteolytic cleavage site; and lacks both an S1 ligand-inducible proteolytic cleavage site and an S2 ligand-inducible proteolytic cleavage site.

In some embodiments, the fusion protein may have an vWF A2 sequence or a variation thereof, an ADAMTS13 cleavage site (which may be described by the consensus sequence HEXXHXXGXXHD SEQ ID NO: 7; Crawley, Blood. 2011 118:3212-21), and an S3 or γ-secretase cleavage site, although many other arrangements exist. In some embodiments, the switch may contain components that are borrowed from Notch. In other embodiments, the switch may not contain components that are from Notch.

For simplicity, BTTSs, including but not limited to chimeric notch receptor polypeptides, are primarily single polypeptide chains. However, BTTSs, including chimeric notch receptor polypeptides, may be divided or split across two or more separate polypeptide chains where the joining of the two or more polypeptide chains to form a functional BTTS, e.g., a chimeric notch receptor polypeptide, may be constitutive or conditionally controlled. For example, constitutive joining of two portions of a split BTTS may be achieved by inserting a constitutive heterodimerization domain between the first and second portions of the split polypeptide such that upon heterodimerization the split portions are functionally joined.

Useful BTTSs that may be employed in the subject methods include, but are not limited to modular extracellular sensor architecture (MESA) polypeptides. A MESA polypeptide comprises: a) a ligand binding domain; b) a transmembrane domain; c) a protease cleavage site; and d) a functional domain. The functional domain can be a transcription regulator (e.g., a transcription activator, a transcription repressor). In some cases, a MESA receptor comprises two polypeptide chains. In some cases, a MESA receptor comprises a single polypeptide chain. Non-limiting examples of MESA polypeptides are described in, e.g., U.S. Patent Publication No. 2014/0234851; the disclosure of which is incorporated herein by reference in its entirety.

Useful BTTSs that may be employed in the subject methods include, but are not limited to polypeptides employed in the TANGO assay. The subject TANGO assay employs a TANGO polypeptide that is a heterodimer in which a first polypeptide comprises a tobacco etch virus (Tev) protease and a second polypeptide comprises a Tev proteolytic cleavage site (PCS) fused to a transcription factor. When the two polypeptides are in proximity to one another, which proximity is mediated by a native protein-protein interaction, Tev cleaves the PCS to release the transcription factor. Non-limiting examples of TANGO polypeptides are described in, e.g., Barnea et al. (Proc Natl Acad Sci USA. 2008 Jan. 8; 105(1):64-9); the disclosure of which is incorporated herein by reference in its entirety.

Useful BTTSs that may be employed in the subject methods include, but are not limited to von Willebrand Factor (vWF) cleavage domain-based BTTSs, such as but not limited to e.g., those containing a unmodified or modified vWF A2 domain. A subject vWF cleavage domain-based BTTS will generally include: an extracellular domain comprising a first member of a binding pair; a von Willebrand Factor (vWF) cleavage domain comprising a proteolytic cleavage site; a cleavable transmembrane domain and an intracellular domain. Non-limiting examples of vWF cleavage domains and vWF cleavage domain-based BTTSs are described in Langridge & Struhl (Cell (2017) 171(6):1383-1396); the disclosure of which is incorporated herein by reference in its entirety.

Useful BTTSs that may be employed in the subject methods include, but are not limited to chimeric Notch receptor polypeptides, such as but not limited to e.g., synNotch polypeptides, non-limiting examples of which are described in PCT Pub. No. WO 2016/138034, U.S. Pat. Nos. 9,670,281, 9,834,608, Roybal et al. Cell (2016) 167(2):419-432, Roybal et al. Cell (2016) 164(4):770-9, and Morsut et al. Cell (2016) 164(4):780-91; the disclosures of which are incorporated herein by reference in their entirety. The “SNIPR” switch is another example of a BTTS (see Zhu et al 2022 Cell. 185: 1431-1443 and WO2021061856), although others exist (e.g., US20200331985) and/or can be readily designed.

Expression of the BTTS in the cell may be constitutive or inducible, e.g., by binding of another BTTS to an antigen on another cell. As such, in some embodiments, the second promoter may be constitutive in the cell. For example, the second promoter may be a CMV, EF-1, hPGK or RPBSA promoter, although many other choices are available.

In any embodiment, the BTTS may comprise: i. an extracellular binding domain that binds to EGFRvIII or the brain-specific marker, ii. an optional force sensing region, iii. a transmembrane domain, iv. one or more force-dependent cleavage sites that are cleaved when the force sensing region is activated, and v. an intracellular domain comprising a transcriptional activator, where binding of the extracellular binding domain to the EGFRvIII or the brain-specific marker induces proteolytic cleavage of the one or more force-dependent cleavage sites to release the transcriptional activator, and wherein the released transcriptional activator induces transcription of the first coding sequence which, in turn, results in expression of the CAR.

Examples of transcriptional activators that can be part of the fusion protein are numerous and include artificial transcription factors (ATFs) such as, e.g., Zinc-finger-based artificial transcription factors (including e.g., those described in Sera T. Adv Drug Deliv Rev. 2009 61(7-8):513-26; Collins et al. Curr Opin Biotechnol. 2003 14(4):371-8; Onori et al. BMC Mol Biol. 2013 14:3. In some cases, the transcriptional activator may contain a GAL4 DNA binding domain, which binds to the Gal4 responsive UAS, which has been well characterized in the art. Examples of suitable transcriptional activators include GAL4-VP16 and GAL4-VP64, although many others could be used. As would be appreciated, the identity of the transcription activators may vary. In some embodiments, the transcription factor may have a DNA binding domain that binds to a corresponding promoter sequence and an activation domain. In many embodiments, the DNA binding domain transcription factor may be independently selected from Gal4-, LexA-, Tet-, Lac-, dCas9-, zinc-finger- and TALE-based transcription factors. TALE- and CRISPR/dCas9-based transcription factors are described in Lebar (Methods Mol Biol. 2018 1772: 191-203), among others. The binding sites for such domains are well known or can be designed at will. The transcriptional activator can have any suitable activation domain, e.g., VP16, VP64, Ela, Sp1, VP16, CTF, GAL4 among many others. The first promoter will contain one or more copies of a binding site for the transcriptional activator, e.g., a GAL4 UAS.

As can be seen from the figures, in any embodiment, the nucleic acid is a double stranded nucleic acid in which the CAR and BTTS are encoded by the top strand of the recombinant nucleic acid.

The cell-surface marker or protein in the extracellular matrix to which the BTTS binds may be tissue-specific, organ-specific or disease-specific (e.g., cancer-specific), many of which are known. Likewise, the cell-surface marker to which the immune receptor (e.g., CAR) binds may be tissue-specific, organ-specific or disease-specific (e.g., cancer-specific), many of which are known.

### Vectors and Particles

In any embodiment, the recombinant nucleic acid may be in a vector, such as a viral vector, e.g., an adenoviral vector, a retroviral vector, a lentiviral vector, an adeno-associated virus vector, an herpes simplex virus vector, etc. Replication-defective viruses can also be advantageous. Some vectors become incorporated into the nuclear genome of the host cell, whereas others do not. The use of viral vectors (particularly retroviral and lentiviral vectors) for expressing CARs in immune cells is known (see, e.g., Lanne et al Viruses. 2021 13: 1528) and Moco et al Methods Mol Biol 2020 2086: 69-76 among many others).

A viral particle comprising the viral vector is also provided. In these embodiments, the particle may be prepared by encapsidating the vector in a viral particle. Numerous methods are known in the art for production of viral particles (see, e.g., Tiscorna Nat Protoc. 2006 1:241-5, among many others).

### Cells and Methods of Treatment

Suitable host cells include cytotoxic cells, including CD8+ T cells, natural-killer (NK) cells, and neutrophils, which cells are capable of mediating cytotoxicity responses. Other immune cells, e.g., immunosuppressing cells such as CD8+ T cells could also be used. The recombinant nucleic acid may integrated into the nuclear genome of the cell or not integrated into the nuclear genome of the cell. In some cases, the introduction of the subject nucleic acids and/or genetic modification is carried out ex vivo. For example, a T lymphocyte, a stem cell, or an NK cell may be obtained from an individual; and the cell obtained from the individual is modified to express components of a circuit of the present disclosure. The modified cell can thus be redirected to one or more antigens of choice, as defined by the one or more antigen binding domains present on the introduced components of the circuit. In some cases, the modified cell is modulated ex vivo. In other cases, the cell is introduced into (e.g., the individual from whom the cell was obtained) and/or already present in an individual; and the cell is modulated in vivo, e.g., by administering a nucleic acid or vector to the individual in vivo. Alternatively, allogeneic immune cells may be used.

As summarized above, the present construct and cell find use in delivering a treatment to a subject for a disease, e.g., disease is cancer, an autoimmune disease, fibrosis, a neurodegenerative disease, diabetes, or an infectious disease. In some cases, the first and second antigens (and the third antigen, if the therapeutic protein is antigen-specific) may be antigens that are expressed by (synthesized by) a cancer cell, i.e., cancer cell-associated antigens. The cancer cell associated antigens can be antigens associated with, e.g., a breast cancer cell, a B cell lymphoma, a pancreatic cancer, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a medulloblastoma, a colorectal cancer cell, etc.

Cancer-specific cell surface/extracellular matrix antigens are numerous and include, but are not limited to:

CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin. Suitable antigen pairs or triples may be identified using the method of Orentas (Front Oncol. 2017; 7: 173) or alternatively, any of the antigen pairs/triples listed in PCT/US19/60357, could be used.

In addition, the present method can be used to treat brain cancers other than glioblastoma, including, but not limited to medulloblastoma, diffuse midline glioma (previously called DIPG), ependymoma, craniopharyngiom, embryonal tumor (previously known as PNET), pineoblastoma, brainstem glioma, choroid plexus carcinoma or germ cell tumor, or one or more adult brain tumors, e.g., pituitary adenoma, acoustic neuroma (also known as vestibular schwannoma), meningioma, oligodendroglioma, haemangioblastoma, CNS lymphoma, non-GBM (or low grade) astrocytoma, a tumor with unknown cells (i.e., an unspecified glioma), Alzheimer's disease, stroke, brain and spinal cord injury, brain cancer, HIV infection in the brain, ataxia-producing disorders, amyotrophic lateral sclerosis (ALS), Huntington disease, childhood inborn genetic errors affecting the brain, Parkinson's disease, and multiple sclerosis. Such treatments may include obtaining a desired effect with respect to at least one disease cell type (or subpopulation thereof) in the brain.

In addition, the present cells can be used to treat inflammatory diseases, organ transplant rejection, and a variety of other diseases, including infections.

In some embodiments, the disease may be a cancer from a non-brain or CNS tissue that has metastasized to the brain. Brain metastases can develop from any type of cancer. The most common types of cancer that spread to the brain are breast cancer, lung cancer, kidney cancer, melanoma, coon cancer, and thyroid cancer. If the solid tumor is a carcinoma, in certain embodiments, the carcinoma is a basal cell carcinoma, squamous cell carcinoma, renal cell carcinoma, ductal carcinoma in situ (DCIS), invasive ductal carcinoma, or adenocarcinoma. Cancers that can be treated with a method include any cancer that can be targeted by CAR-T cells, including, but not limited to carcinomas, sarcomas, melanoma, leukemias, lymphomas and multiple myeloma. Cancers that can be treated with a method include solid tumors, and cancers that begin in blood-forming tissue, i.e., hematological cancers such as leukemias, lymphomas and multiple myeloma. Cancers that can be treated with a method include metastatic cancers. Carcinomas that can treated by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma. Sarcomas that can be treated by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas. Other solid tumors that can be treated by a method disclosed herein include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In any embodiment, the BTTS may recognize a tissue-specific marker (e.g., a marker that is specific for brain, lung, pancreas, colon, liver, skin, etc.) and the therapeutic protein (e.g., a CAR) may be activated by a tumor associated antigen. In these embodiments, the cytotoxic activity of the cell may be confined to a particular cell.

The subject methods may include introducing into a subject in need thereof, a population of the cells described above. The introduced cells may be immune cells, including e.g., myeloid cells or lymphoid cells. In other cases, the introduced cells are not immune cells.

In some instances, the methods described herein may be used before or after a conventional therapy. For example, the methods described herein may be used as an adjuvant therapy, e.g., after a subject has seen improvement from a conventional therapy, or may be used when a subject has not responded to a conventional therapy. In some instances, the methods described herein may be used prior to an additional therapy, e.g., to prepare a subject for an additional therapy, e.g., a conventional therapy as described herein.

Standard therapies include surgery (e.g., surgical removal of cancerous tissue), radiation therapy, chemotherapeutic treatment, antibody treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Antibody therapies include, but are not limited to, naked antibodies, e.g., trastuzumab (Herceptin), bevacizumab (Avastin™), cetuximab (Erbitux™), panitumumab (Vectibix™), Ipilimumab (Yervoy™), rituximab (Rituxan), alemtuzumab (Lemtrada™), Oregovomab (OvaRex™), Lambrolizumab (pembrolizumab, MK-3475, Keytruda™), ranibizumab (Lucentis™) etc., and conjugated antibodies, e.g., conjugated antibodies of those listed above and the like.

In some instances, the methods of the instant disclosure may be used without any additional conventional therapy including e.g., where the method described herein is the sole method used to treat the subject.

Determining when combination therapies, e.g., involving the administration of one or more agents that ameliorates one or more side effects of a therapy described herein or involving the administration of one or more agents that enhances a therapy described herein, are indicated and the specifics of the administration of such combination therapies are within the skill of the relevant medical practitioner. In some instances, dosage regimens and treatment schedules of combination therapies may be determined through clinical trials.

As will be readily understood, the methods of treating described herein may, in some instances, be combined with one or more conventional treatments. For example, in the case of oncology for GBM, the methods described herein may, in some instances, be combined with a conventional GBM therapy including but not limited to e.g., conventional chemotherapy, conventional radiation therapy, conventional immunotherapy, surgery, etc. Also as described above, in some instances, the methods of treating described herein may be employed following conventional therapy, e.g., to treat a heterogeneous GBM that is refractory to a conventional therapy, to treat a heterogeneous GBM that is recurrent after a conventional therapy, to treat a subject for MRD following conventional therapy, and the like.

In some instances, the methods described herein may be used before or after a conventional therapy. For example, the methods described herein may be used as an adjuvant therapy, e.g., after a subject has seen improvement from a conventional therapy, or may be used when a subject has not responded to a conventional therapy. In some instances, the methods described herein may be used prior to an additional therapy, e.g., to prepare a subject for an additional therapy, e.g., a conventional therapy as described herein.

Standard GBM therapies include surgery (e.g., surgical removal of cancerous tissue), radiation therapy, chemotherapeutic treatment, antibody treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Antibodies suitable for use in, or under investigation for, GBM treatment include, but are not limited to, naked antibodies, e.g., trastuzumab (Herceptin), bevacizumab (Avastin™) cetuximab (Erbitux™), panitumumab (Vectibix™), Ipilimumab (Yervoy™), rituximab (Rituxan), alemtuzumab (Lemtrada™), Oregovomab (OvaRex™), Lambrolizumab (pembrolizumab, MK-3475, Keytruda™), ranibizumab (Lucentis™) etc., and conjugated antibodies, e.g., conjugated antibodies of those listed above and the like.

Conventional cancer therapies also include targeted therapies for cancer including but not limited to e.g., Bevacizumab (Avastin) targeting VEGF ligand (approved for use in Glioblastoma) and the like.

In some instances, the methods of the instant disclosure may be used without any additional conventional therapy including e.g., where the method described herein is the sole method used to treat the subject. For example, in the case of oncology, the methods described herein may, in some instances, be the sole method used to treat the subject for a GBM, including e.g., a primary GBM, a recurrent GBM, and the like.

Determining when combination therapies, e.g., involving the administration of one or more agents that ameliorates one or more side effects of a therapy described herein or involving the administration of one or more agents that enhances a therapy described herein, are indicated and the specifics of the administration of such combination therapies are within the skill of the relevant medical practitioner. In some instances, dosage regimens and treatment schedules of combination therapies may be determined through clinical trials.

### Populations of T Cells

A population of the T cells is also provided. In some embodiments, these cells may be present in vitro and may be progenitors of primary T cells that have been genetically modified to have the recombinant nucleic acid. In some embodiments, the cell may contain a nucleic acid encoding an immune receptor, as discussed above. As noted above, in some embodiments T cells may be genetically modified to be allogeneic in a human host. In these embodiments, the cells may be frozen. The population may comprise any number of the CAR-T cells (e.g. 100,000-1 Bn cells). However, in some embodiments, the population may contain 100,000-50M of the cells.

In some embodiments, the harvested cells may be cryopreserved, where the term “cryopreserved” refers to cells that have been preserved or maintained by cooling to low sub-zero temperatures, such as 77 K or −196 deg. C. (the boiling point of liquid nitrogen). At these low temperatures, any biological activity, including the biochemical reactions that would lead to cell death, is effectively stopped. Useful methods of cryopreservation and thawing cryopreserved cells, as well as processes and reagents related thereto, include but are not limited to e.g., those described in U.S. Pat. Nos. 10,370,638; 10,159,244; 9,078,430; 7,604,929; 6,136,525; and 5,795,711, the disclosures of which are incorporated herein by reference in their entirety. In contrast, the term “fresh”, as used herein with reference to cells, may refer to cells that have not been cryopreserved and, e.g., may have been directly obtained and/or used (e.g., transplanted, cultured, etc.) following collection from a subject or organ thereof.

Harvested therapeutic cell populations produced by the methods as described herein and therapeutic or pharmaceutical compositions thereof may be present in any suitable container (e.g., a culture vessel, tube, flask, vial, cryovial, cryo-bag, etc.) and may be employed (e.g., administered to a subject) using any suitable delivery method and/or device. Such populations of cells and pharmaceutical compositions may be prepared and/or used fresh or may be cryopreserved. In some instances, populations of therapeutic cells and pharmaceutical compositions thereof may be prepared in a “ready-to-use” format, including e.g., where the therapeutic cells are present in a suitable diluent and/or at a desired delivery concentration (e.g., in unit dosage form) or a concentration that can be readily diluted to a desired delivery concentration (e.g., with a suitable diluent or media). Populations of therapeutic cells and pharmaceutical compositions thereof may be prepared in a delivery device or a device compatible with a desired delivery mechanism or the desired route of delivery, such as but not limited to e.g., a syringe, an infusion bag, or the like.

In some instances, the present disclosure provides one or a plurality of cell therapy doses, e.g., each contained in suitable container. Cell therapy doses may be generated through a variety of methods. Aliquoting expanded populations of therapeutic cells into cell therapy doses may be performed by a variety of means.

In certain embodiments, the compositions may include the therapeutic cells present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions.

A population may include a therapeutically effective amount of the cells. By “therapeutically effective amount” it is meant a number of cells sufficient to produce a desired result, e.g., an amount sufficient to affect beneficial or desired therapeutic (including preventative) results, such as a reduction in a symptom of a disease or disorder associated, e.g., with the target cell or a population thereof, as compared to a control. An effective amount can be administered in one or more administrations.

A “therapeutically effective amount” of such cells may vary according to factors such as the disease state, age, sex, and weight of the subject, and the ability of the cells to elicit a desired response in the subject. A therapeutically effective amount is also one in which any toxic or detrimental effects of the cells are outweighed by the therapeutically beneficial effects. The term “therapeutically effective amount” includes an amount that is effective to “treat” a subject (e.g., a patient). When a therapeutic amount is indicated, the precise amount of the compositions contemplated in particular embodiments, to be administered, can be determined by a physician in view of the specification and with consideration of individual differences in age, weight, tumor size, extent of infection or metastasis, and condition of the patient (subject). In certain embodiments, a therapeutically effective amount of T cells may be 100,000-50M of the T cells. However, in other embodiments, a therapeutically effective amount of T cells may be 50M-500M cells.

The cells of the present disclosure can be incorporated into a variety of formulations for therapeutic administration. More particularly, the cells of the present disclosure can be formulated for administration by combination with appropriate excipients, diluents and/or the like.

Formulations of the cells suitable for administration to a patient (e.g., suitable for human administration) are generally sterile and may further be free of detectable pyrogens or other contaminants contraindicated for administration to a patient according to a selected route of administration.

The cells may be formulated for parenteral (e.g., intravenous, intra-arterial, intraosseous, intramuscular, intracerebral, intracerebroventricular, intrathecal, subcutaneous, etc.) administration, or any other suitable route of administration. In any embodiment, the cells may be administered intracranially, e.g., by direct injection into a tumor.

An aqueous formulation of the cells may be prepared in a pH-buffered solution, e.g., at a pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

In some embodiments, a composition includes cells of the present disclosure, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Cell Manufacture Methods

A cell manufacture method is also provided. In some embodiments, the method may comprise expanding T cells comprising the recombinant nucleic acid ex vivo to produce expanded T cells, and harvesting the expanded T cells to produce a T cell population, where the term “harvesting” is intended to refer to a step in which the cells are removed from the container(s)/bioreactor(s) in which the cells were expanded. In some embodiments, the cells may be concentrated if desired, e.g., by centrifugation, a suitable cell separation technique (e.g., magnetic beads), and/or the like.

The cells may be made by any method, e.g., by introducing the recombinant nucleic acid into the T cells. The general procedure for manufacturing CAR-T cells typically includes isolation of peripheral blood mononuclear cells (PBMCs). Next, PBMCs or T cells that have been further enriched from PBMCs are stimulated with antibody-coated beads (e.g. Dynabeads) or plate-bound antibodies to induce T cell activation and then genetically modified using lentiviral vectors, gamma-retroviral vectors, or other delivery methods, to express the cell surface CAR molecule. Subsequently, these engineered T cells are expanded in culture in the presence of one or more cytokines (e.g., IL-2, IL-15, IL-7 or any combination thereof (e.g., IL-2 and IL-15 or IL-7 and IL-15)) for several days to reach the required cell numbers for either experimental testing or clinical treatment.

In some embodiments, the T cells may be harvested within 3-8 days of the initiation of cell expansion (e.g., on day 3, day 4, day 5, day 6, day 7 or day 7), which is earlier than conventional procedures (which typically require 10-14 days of cell expansion). In these embodiments, the harvested T cell population may comprise 100,000-50M of the CAR-T cells, however more or less cells can be used. In other embodiments, the T cells may be harvested within 8-16 days of the initiation of cell expansion, in which case the population may comprise 50M-1 Bn cells.

A cell population manufactured according to this method is also provided.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Overview of E-SYNC Therapeutic T Cells and Generation of p885 E-SYNC Clinical Single Lentiviral Transfer Vector from the Original Two Vector Research-Grade Design

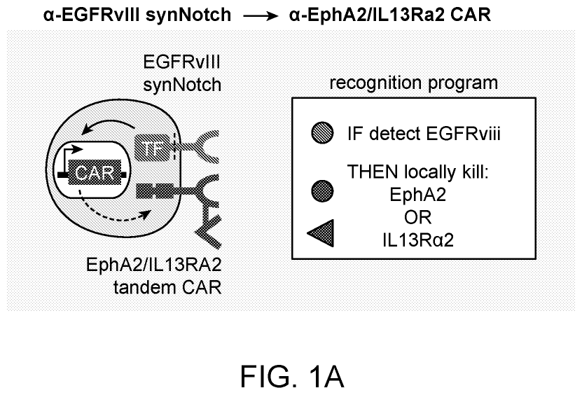

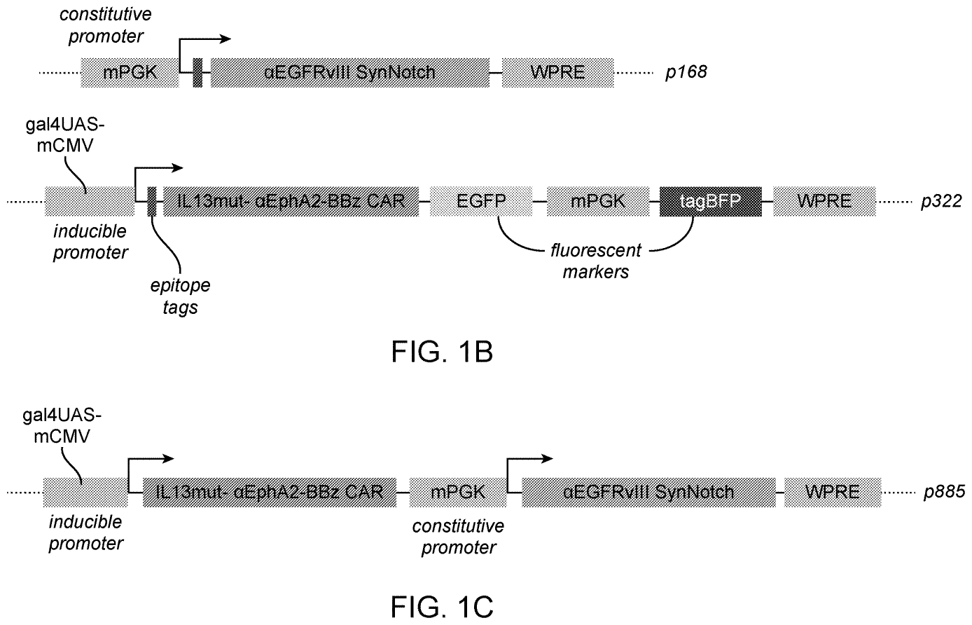

FIG. 1A shows a diagram of a “prime and kill” E-SYNC T cell circuit. E-SYNC are primed when exposed to cells displaying the GBM specific EGFRvIII marker, which triggers activation of the synNotch receptor (in orange) to activate expression of the anti-EphA2/IL13Rα2 tandem CAR. Once prime, E-SYNC T cells are capable of killing GBM cells that naturally overexpress these markers.

FIG. 1B shows the EGFRvIII-primed synNotch-CAR GBM therapeutic circuit demonstrated in Choe et al (Sci. Transl. Med. 2021 13: cabe7378) utilized a dual lentiviral vector system, composed of a synNotch-expressing vector (p168) and a payload vector (p322) that encoded the CAR under a synNotch-inducible promoter. While effective in all experiments described in the publication, this dual vector system may be potentially unsuitable for manufacturing T cells for clinical application given its cost-inefficient dual vector design as well as use of epitope tags (purple) and fluorescent markers.

FIG. 1C shows a diagram of clinical E-SYNC single vector design (p885). The inducible CAR transgene (in blue) was placed in tandem, upstream of the constitutive synNotch transgene. All superfluous DNA elements (tags, markers) were removed and parts of the coding sequence were optimized for improved expression and viral titer. The entire vector payload was subcloned into a 3^{rd} generation lentiviral backbone compatible with cGMP viral manufacturing.

### Example 2

### Examples of Single Lentiviral Vector Configurations for BTTS Driven CAR Circuits

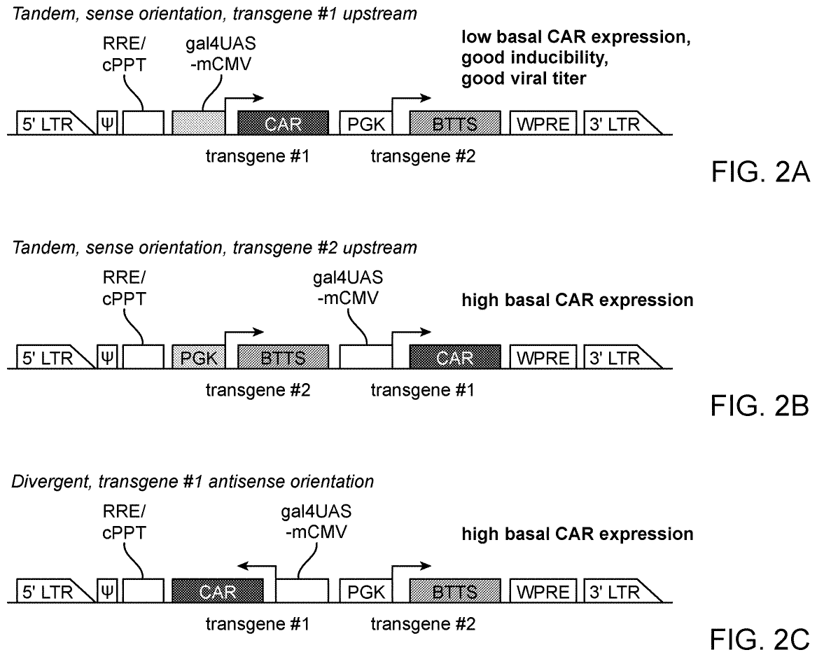

FIGS. 2A-2C show several different lentiviral vector configurations were explored where transgenes were either placed in tandem (A and B) or in divergent orientation (C). These different configurations were evaluated for i) strong BTTS (e.g. synNotch) driven CAR expression and ii) low basal CAR expression in the absence of BTTS stimulation. Observations are summarized below. Given that detectable basal CAR expression in therapeutic cells presented a significant risk for adverse effects, only design A was considered for E-SYNC and B-SYNC single vector development.

### Example 3

### E-SYNC T Cells Prepared with p885 Single Vector Show EGFRvIII-Driven CAR Expression and Kill GBM Tumor Cells In Vitro Similarly to Reference Dual Vector Cells

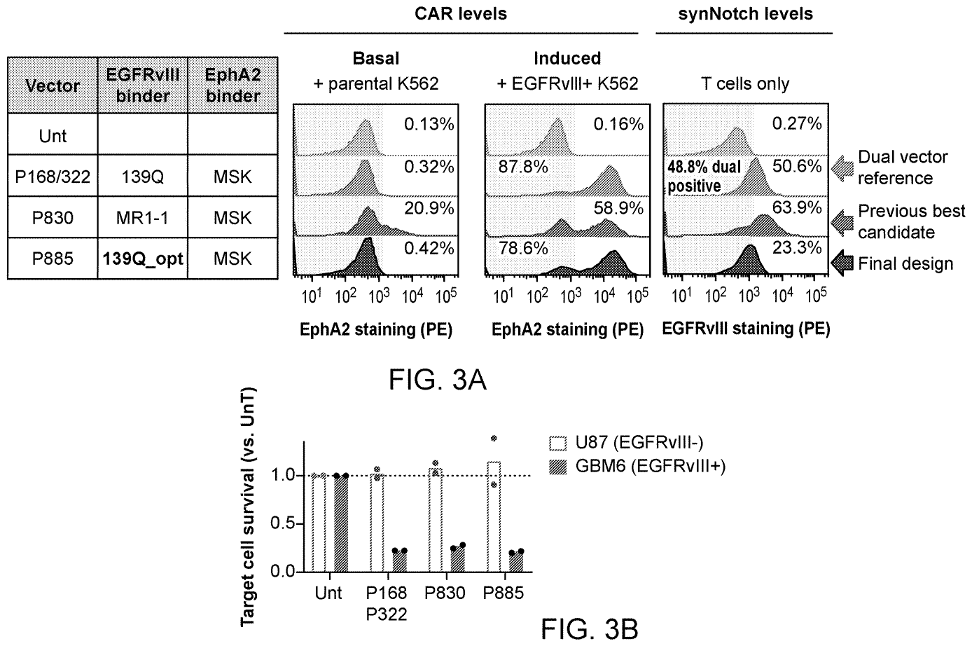

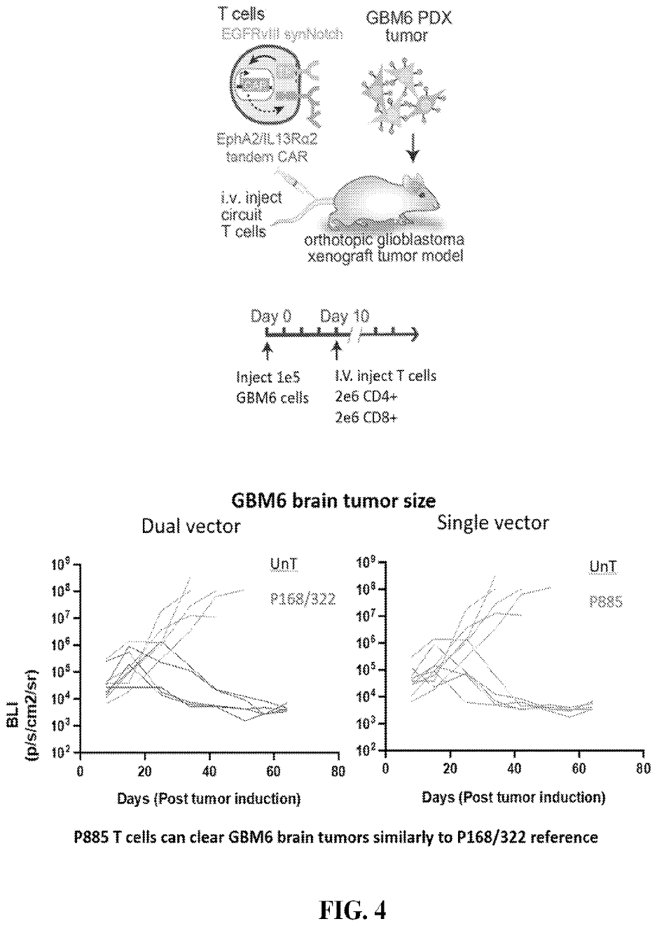

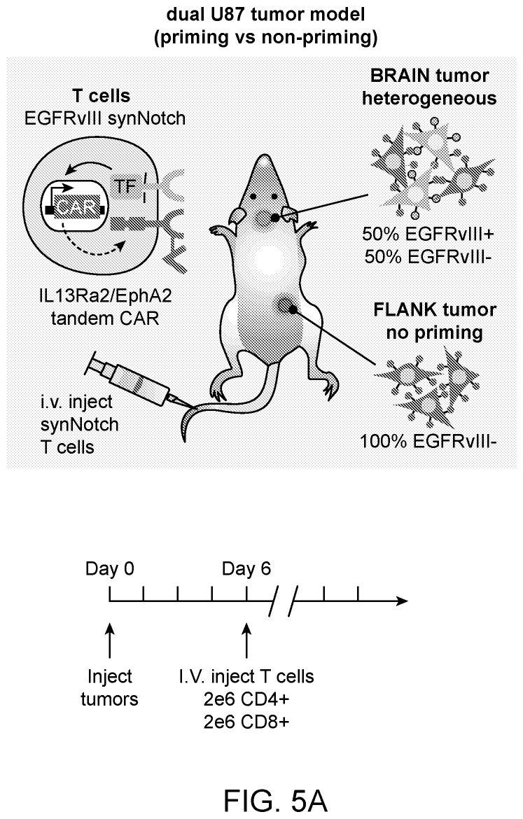

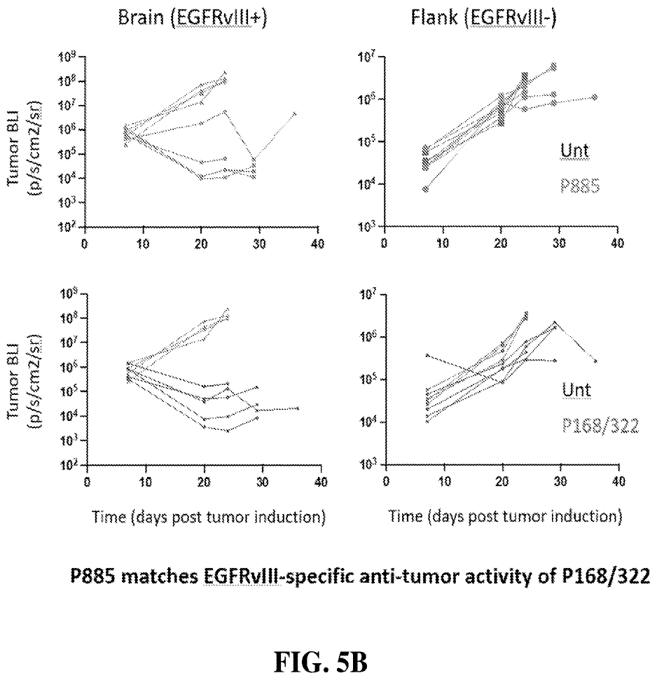

FIG. 3A shows that P885 T cells can activate CAR expression in response to EGFRvIII exposure to similar levels as dual vector reference cells while maintaining low basal CAR expression in the absence of stimulation. CD8+ T cells were transduced with dual vector design (P168 and P322) as well as two finalist single vector designs (P830 and P885). Differences in binder composition is listed on left table. T cells were tested for their ability to induce CAR expression. 50k T cells were co-cultured with K562 cells at 1:1 ratio for 48 h and then stained for CAR levels (middle two panels) or synNotch (right panel). Basal expression of CAR was evaluated with parental K562s, while synNotch-CAR activation was measured with EGFRvIII+K562 cells.

FIG. 3B illustrate that killing assay measuring the capability of CD8+ T cells transduced with P168/322, P830 or P885 of clearing GBM cells shows that single vector designs can match dual vector reference. 50k T cells were challenged with either U87 MG (EGFRvIII negative) or GBM6 PDX (EGFRvIII+) cells in co-culture for 48 h (E:T=1). Target cell survival was analyzed by flow cytometry and normalized to control wells containing untransduced T cells. Data shown for two technical replicates.

### Example 4

### E-SYNC T Cells Prepared with p885 Clinical Vector are Effective in Clearing EGFRvIII+GBM Patient Derived Xenograft Brain Tumors in Mice

T cells transduced with single P885 candidate vector can clear the GBM6 PDX tumors similarly to dual vector reference cells. NCG mice were orthotopically implanted with GBM6 patient derived xenograft tumor cells in the brain and subsequently received an intravenous dose of CD4+ and CD8+ T cells transduced with either P168/P322 dual vector, P885 clinical single vector candidate or untransduced T cells (timeline and doses listed below). Tumor size measurements were taken by bioluminescence live imaging of effLuc marker using an IVIS Spectrum imager every 7 days post tumor induction. Tumor size traces for individual mice from each group are plotted below.

### Example 5

E-SYNC T Cells Transduced with p885 Single Vector can Discriminate Between Brain Vs. Flank Tumors In Vivo Based on Tumor EGFRvIII Expression

T cells prepared with single P885 candidate vector clear the EGFRvIII+ brain tumor but do not control the EGFRvIII− negative tumor in the flank, demonstrating similar spatial control of CAR activity in vivo to the published dual vector reference design. NCG mice were orthotopically implanted in the brain with a priming U87 MG tumor (1:1 mixture of EGFRvIII+ and EGFRvIII− cells) and a non-priming U87 tumor in the flank (EGFRvIII− U87 MG cells). The mice subsequently received an intravenous dose of CD4+ and CD8+ T cells transduced with either P168/P322 dual vector, P885 clinical single vector candidate or untransduced T cells (timeline and doses listed below). Tumor size measurements were taken by bioluminescence live imaging of effLuc marker using an IVIS Spectrum imager every 7 days post tumor induction. Tumor size traces for individual mice from each group are plotted below.

### Example 6

Overview of BCAN-Primed SynNotch-CAR Circuit (B-SYNC), Composition of pB-SYNC1 Single Lentiviral Transfer Vector and Tumor Cell Killing Performance of B-SYNC T Cells

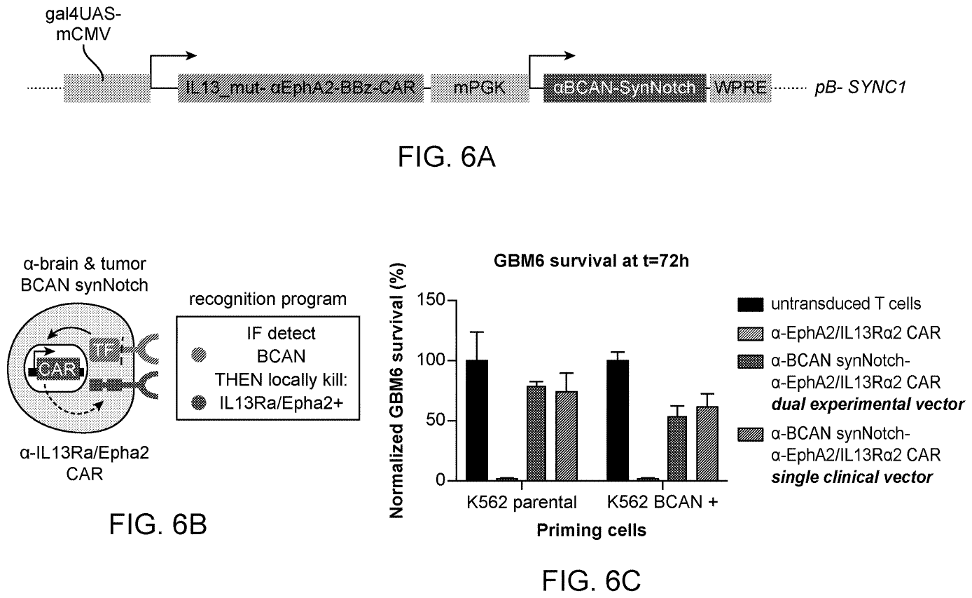

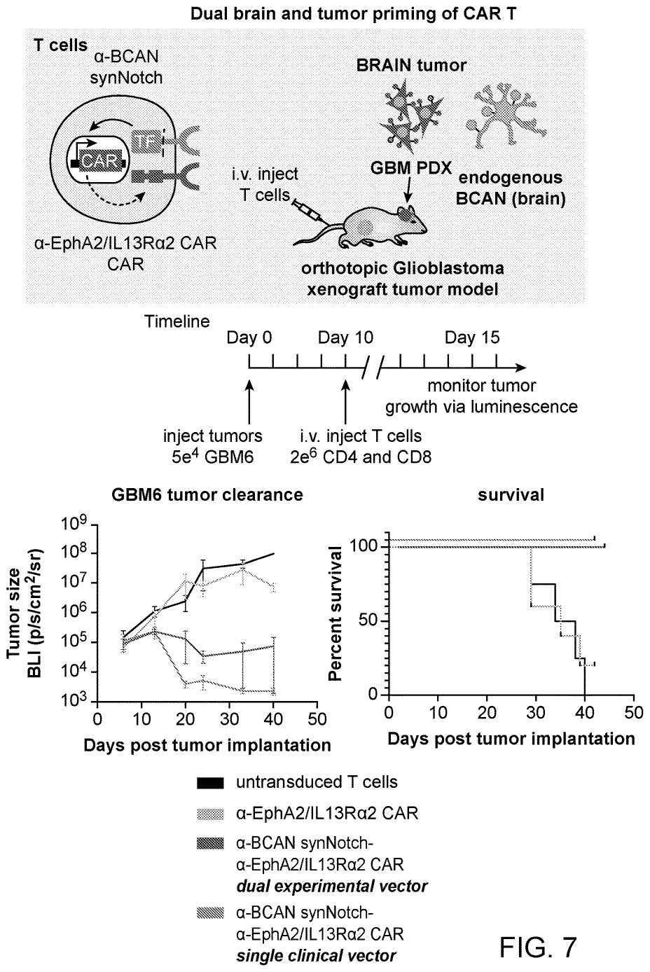

FIG. 6A provides an overview of pB-SYNC1 vector design to produce BCAN-primed SynNotch-CAR (B-SYNC) T cells. Design was based on the p885 vector, with the difference of the anti-BCAN binder expressed as part of the synNotch receptor.

FIG. 6B is a diagram of B-SYNC therapeutic T cell. Unlike E-SYNC that requires the presence of GBM-specific EGFRvIII antigen, B-SYNC cells are primed by exposure to the brain-specific ligand Brevican (BCAN). This broadens the resulting expression of the dual affinity CAR to the entire central nervous system, which enables treatment of a wider range of EGFRvIII-negative GBM patients.

FIG. 6C shows that T cells transduced with pB-SYNC1 single vector outperform dual vector reference cells in this in vitro co-culture killing assay of GBM6 PDX. Assay was prepared using T cells transduced with either B-SYNC clinical single vector, dual vector reference, constitutive dual affinity CAR or untransduced control. T cells and GBM6 were mixed in a three-way co-culture with either parental or BCAN+ K562 cells in a 1:1:1 ratio to provide circuit priming. After 72 h in culture, the remaining target GBM6 cells were analyzed by flow cytometry and normalized to wells containing untransduced T cells. Average of two experimental replicates shown with standard deviation as error bars.

### Example 7

B-SYNC T Cells Prepared with pB-SYNC1 Clinical Vector are Effective in Clearing GBM Patient Derived Xenograft Tumors in Mice and Improving Survival.

T cells transduced with pB-SYNC1 single clinical vector candidate can clear GBM6 brain tumors in mice similarly to dual vector reference T cells. NCG mice orthotopically were implanted with GBM6 patient derived xenograft tumor cells in the brain and subsequently received an intravenous dose of CD4+ and CD8+ T cells transduced with either pB-SYNC1 clinical single vector candidate, reference dual vector, vector expressing CAR constitutively or untransduced T cells (timeline and doses listed below). Tumor size measurements were taken by bioluminescence live imaging of effLuc marker using an IVIS Spectrum imager every 7 days post tumor induction. Average tumor size traces for individual mice from each group are plotted below. Kaplan-Meier survival curves for the different mice groups shown on the right.

### Example 8

### Single Vector Designs

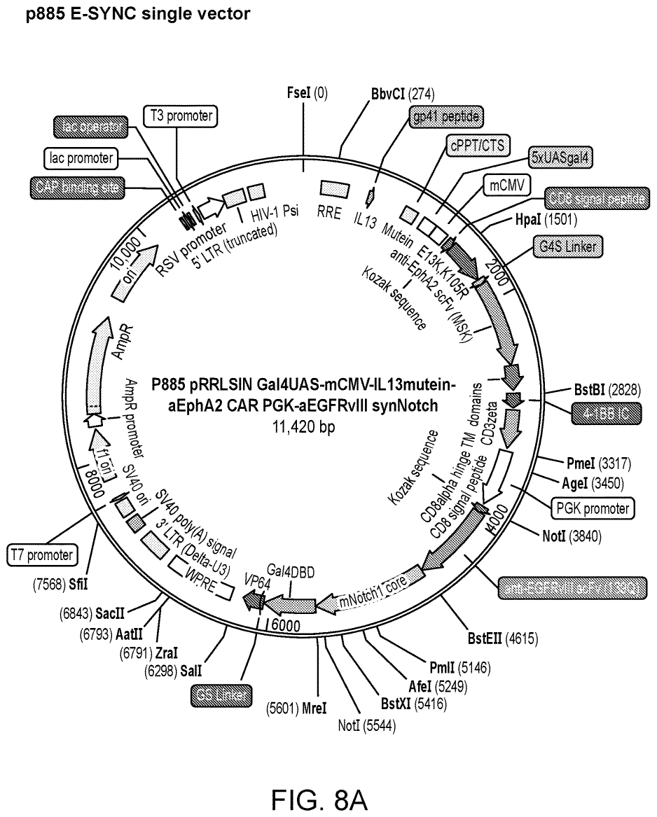

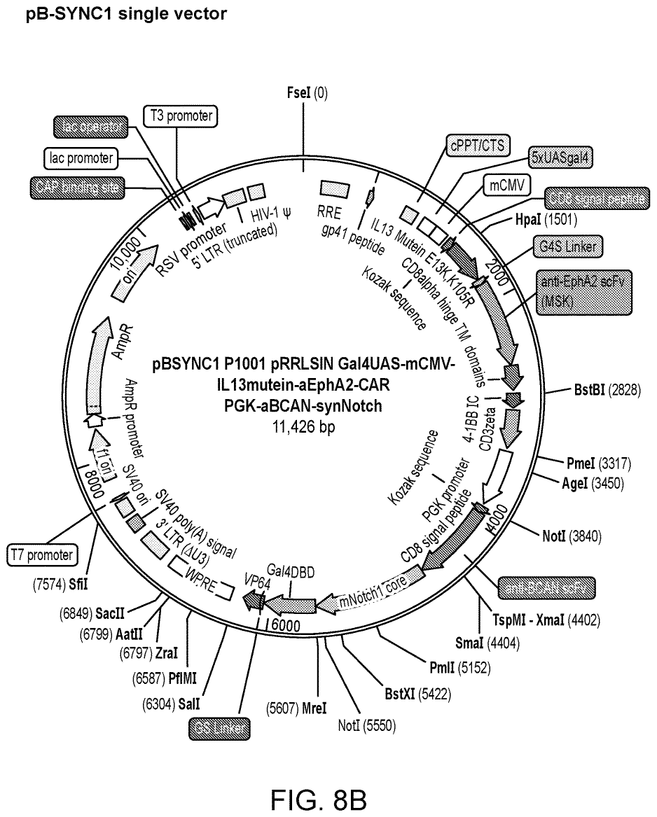

FIGS. 8A and 8B show plasmid maps for p885 and pB-SYNC1 vectors as described above.

FIG. 9A shows an initial tandem, which is suitable for first generation therapeutic BTTS-controlled CAR expression circuits but may be limited to a single protein payload. In order to overcome the challenges of the tumor microenvironment and enhance T cell function in vivo, expression of additional protein payloads together with CAR may be advantageous.

FIG. 9B illustrates a tandem vector design can be modified to accommodate more than one regulated protein through the use of 2A peptides or internal ribosome entry sites (IRES) to create multi-cistronic transgenes that can produce multiple proteins under BTTS control.

FIG. 9C shows a alternative design is to utilize a bi-directional BTTS-regulated synthetic promoter composed of a BTTS transcription factor binding array flanked by two minimal promoters in divergent origentation. This synthetic bidirectional promoter can induce expression of two separate transgenes under the control of a single BTTS. This strategy can also be combined with bicistronic elements (FIG. 9B) to drive expression of more than 2 protein payloads under BTTS control.

FIG. 9D: Primary human T cells were produced with either (1) unidirectional system for induction of a CAR and IL-2 linked by 2a elements and triggered by a binding triggered transcriptional switch in tandem or (2) bidirectional system for induction of CAR and IL-2 using distinct minimal promoters in opposing orientations flanking the binding triggered transcriptional switch transcription factor binding site. Myc tagged BTTS receptor was triggered using anti-myc beads and CAR induction measured by flow cytometry for epitope tag. Bidirecitonal promoter showed far more robust induction of CAR expression.

FIG. 9E: As in D, primary human T cells were engineered with unidirectional or bidirectional BTTS circuits for production of CAR and IL-2. Myc tagged BTTS receptor was triggered using anti-myc beads and IL-2 induction measured by ELISA 24 hours after addition of beads. Bidirecitonal promoter showed far more robust induction of IL-2 production.

### Example 9

### Use of the CMV Promoter

Modifying 5′ LTR to use CMV promoter enhances titer of large vector designs using state of the art 3^{rd} generation lentiviral system for clinical use.

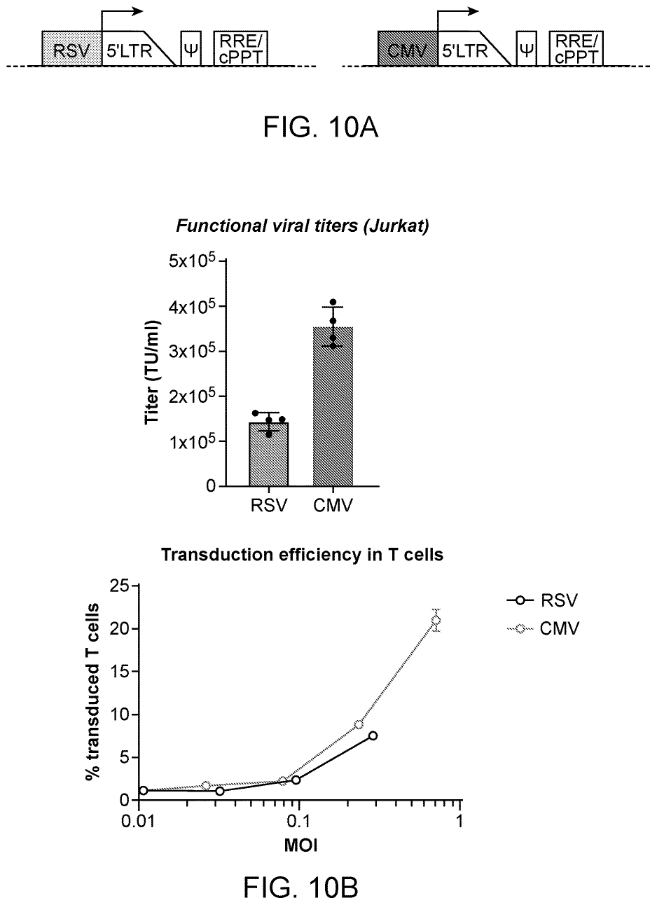

FIG. 10A: scalability of pRRL 3^{rd} gen lentiviral backbone (containing a 5′ chimeric LTR with Rous Sarcoma Virus promoter) is limited when combined with large (>7 kb) DNA constructs, resulting in clinical grade virus with low titer and poor performance with T cells. We have modified the 5′ LTR with alternative constitutive promoters to enhance viral titer and packaging efficiency (including CMV, EF1a and SFFV promoters). This chimeric LTR/CMV promoter has the following sequence:

(SEQ ID NO: 8)
AGCTGGAGCTGCAAGCTTTGATCAGACATTGATTATTGACTAGTTATTA
ATAGTAATCAATTACGGGGTCATTAGTTCATAGCCCATATATGGAGTTC
CGCGTTACATAACTTACGGTAAATGGCCCGCCTGGCTGACCGCCCAACG
ACCCCCGCCCATTGACGTCAATAATGACGTATGTTCCCATAGTAACGCC
AATAGGGACTTTCCATTGACGTCAATGGGTGGAGTATTTACGGTAAACT
GCCCACTTGGCAGTACATCAAGTGTATCATATGCCAAGTACGCCCCCTA
TTGACGTCAATGACGGTAAATGGCCCGCCTGGCATTATGCCCAGTACAT
GACCTTATGGGACTTTCCTACTTGGCAGTACATCTACGTATTAGTCATC
GCTATTACCATGGTGATGCGGTTTTGGCAGTACATCAATGGGCGTGGAT
AGCGGTTTGACTCACGGGGATTTCCAAGTCTCCACCCCATTGACGTCAA
TGGGAGTTTGTTTTGGCACCAAAATCAACGGGACTTTCCAAAATGTCGT
AACAACTCCGCCCCATTGACGCAAATGGGCGGTAGGCGTGTACGGTGGG
AGGTCTATATAAGCAGCGCGTTTTGCCTGTACTGGGTCTCTCTGGTTAG
ACCAGATCTGAGCCTGGGAGCTCTCTGGCTAACTAGGGAACCCACTGCT
TAAGCCTCAATAAAGCTTGCCTTGAGTGCTTCAAGTAGTGTGTGCCCGT
CTGTTGTGTGACTCTGGTAACTAGAGATCCCTCAGACCCTTTTAGTCAG
TGTGGAAAATCTCTAGCAGTGGCGCCCGAACAGGGACTT

In this sequence, nucleotides 25-404 and 405-607 are the CMV enhancer and promoter respectively, and nucleotides 622-802 are the truncated 5′ long terminal repeat (LTR) from HIV-1.

FIG. 10B: CMV-modified 5′ LTR lentiviral vectors containing an identical 7.8 kb payload produced higher functional titers (measured in Jurkat cells, left) and improved T cell transduction efficiency when compared with the reference RSV 5′ LTR pRRL vector version.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A recombinant nucleic acid comprising, in order from 5′ to 3′:
(a) a first promoter;
(b) a first coding sequence, wherein the first coding sequence encodes a therapeutic protein and is operably linked to the first promoter;
(c) a second promoter; and
(d) a second coding sequence, wherein the second coding sequence encodes a binding triggered transcriptional switch (BTTS) that is activated by binding to a cell surface antigen or a protein in the extracellular matrix and is operably linked to the second promoter,
wherein:
transfection of recombinant nucleic acid into an immune cell results in expression of the BTTS on the surface of the immune cell and
binding of the expressed BTTS to an antigen on the surface of a target cell or protein in the extracellular matrix activates expression of the therapeutic protein by the immune cell.

2. The recombinant nucleic acid of claim 1, wherein the BTTS is activated by binding to a tissue-specific, disease-specific or organ-specific marker.

3. The recombinant nucleic acid of claim 1, wherein the therapeutic protein is an immune receptor, antibody, cytokine, cytokine receptor or enzyme.

4. The recombinant nucleic acid of claim 1, wherein the therapeutic protein is an immune receptor that is activated by binding to a tissue-specific, disease-specific or organ-specific marker.

5. The recombinant nucleic acid of claim 1, wherein the BTTS comprises:
i. an extracellular binding domain that binds to a cell surface marker or protein in the extracellular matrix,
ii. a transmembrane domain,
iii. one or more cleavage sites that are cleaved when the extracellular binding domain binds to the cell surface marker or protein in the extracellular matrix, and
iv. an intracellular domain comprising a transcriptional activator,
wherein binding of the extracellular binding domain to the cell surface marker or protein in the extracellular matrix induces proteolytic cleavage of the one or more cleavage sites to release the transcriptional activator, and
wherein the released transcriptional activator induces transcription of the first coding sequence.

6. The recombinant nucleic acid of claim 1, wherein the second promoter is constitutive in the immune cell.

7. The recombinant nucleic acid of claim 1, wherein the nucleic acid is double stranded and wherein the therapeutic protein and BTTS are encoded by the top strand of the recombinant nucleic acid.

8. The recombinant nucleic acid of claim 1, wherein:
the first promoter is bi-directional;
the recombinant nucleic acid further comprises the complement of a third coding sequence that is 5′ of the bi-directional promoter; and
binding of the BTTS to an antigen on the surface of a target cell or protein in the extracellular matrix activates expression of the proteins encoded by first and third coding sequences by the immune cell.

9. The recombinant nucleic acid of claim 1, wherein the bidirectional promoter has at least 90% sequence identity to SEQ ID NO: 9.

10. The recombinant nucleic acid of claim 1, wherein the recombinant nucleic acid further comprises the complement of a transcriptional terminator that is 5′ of the complement of the third coding sequence.

11. The recombinant nucleic acid of claim 10, wherein the transcriptional terminator has at least 90% sequence identity to SEQ ID NO: 10.

12. The recombinant nucleic acid of claim 1, wherein:
the recombinant nucleic acid further comprises a third coding sequence that is 3′ of the first coding sequence, before the second promoter;
the first and third coding sequences are separated by an IRES or a 2A coding sequence and
binding of the BTTS to an antigen on the surface of a target cell or protein in the extracellular matrix activates expression of the proteins encoded by first and third coding sequences by the immune cell.

13. The nucleic acid of any of claim 1, wherein the nucleic acid further comprises a chimeric CMV 5′LTR fusion sequence that has at least 90% sequence identity to SEQ ID NO: 8.

14. A lentiviral vector comprising the recombinant nucleic acid of claim 1.

15-18. (canceled)

19. A cell comprising the recombinant nucleic acid of claim 1.

20-21. (canceled)

22. The cell of claim 19, wherein the cell is a cytotoxic immune cell.

23. (canceled)

24. A method of treatment, comprising:
administering a cell of claim 19 to a subject that is in need thereof.

25. The method of claim 24, wherein the subject has an inflammatory disease.

26. The method of claim 24, wherein the subject has cancer.

27. (canceled)

28. A chimeric CMV 5′LTR fusion that has at least 90% sequence identity to SEQ ID NO: 8.
